(19) United States
(12) Patent Application Publication
(10) Pub. No.: US 2006/0174885 A1
(43) Pub. Date: Aug. 10, 2006

(54) Method and related system to control applied pressure in CPAP systems

(75) Inventors: Alonzo C. Aylsworth, Wildwood, MO (US); Lawrence C. Spector, Austin, TX (US)

Correspondence Address: CONLEY ROSE, P.C., P. O. BOX 3267, HOUSTON, TX, 77253-3267, US

(73) Assignee: ACOBA, LLC, Chesterfield, MO (US)

(21) Appl. No.: 11/156,432

(22) Filed: Jun. 20, 2005

Related U.S. Application Data
(60) Provisional application No. 60/650,796, filed on Feb. 8, 2005.

(51) Int. Cl. A62B 7/10; A61M 15/08

(52) U.S. Cl. 128/206.11; 128/207.18

(57) ABSTRACT

A method and related system of controlling therapeutic gas provided to a patient in positive airway pressure applications. Some of the illustrative embodiments may be a method comprising supplying therapeutic gas at a first pressure to a first naris of patient during a current respiratory cycle and a subsequent respiratory cycle, selecting a second pressure based on an attribute of airflow through a second naris of the patient, and supplying therapeutic gas at the second pressure to the second naris during the current respiratory cycle.

This application is related to application Ser. No. 10/851,952 filed May 21, 2004 titled, “Method and System of Individually Controlling Airway Pressure of a Patient's Nares,” which application is incorporated by reference herein as if reproduced in full below. Moreover, this application claims the benefit of provisional application Ser. No. 60/650,796, filed Feb. 8, 2005 titled, “Method and related system to control applied pressure in CPAP systems,” which application is also incorporated by reference herein as if reproduced in full below.

### STATEMENT REGARDING FEDERALLY SPONSORED RESEARCH OR DEVELOPMENT

Not applicable.

### BACKGROUND OF THE INVENTION

1. Field of the Invention

Embodiments of the present invention are directed to positive airway pressure devices and methods, for example continuous positive airway pressure (CPAP) devices. More particularly, some embodiments of the invention are directed to positive airway pressure devices and methods where the flow and/or pressure through each nostril or naris may be individually controlled.

2. Background of the Invention

Sleep disordered breathing is common throughout the population, and some sleep disorder breathing may be attributable to disorders of the respiratory tract. For example, sleep apnea is a situation where a person temporarily stops breathing during sleep. A hypopnea is a period of time where a person's breathing becomes abnormally slow or shallow. In some cases, a hypopnea may precede an apnea event.

Although hypopneas and apneas may have multiple causes, one trigger for these type events may be full or partial blockages in the respiratory tract. In particular, in some patients the larynx may collapse due to forces of gravity and/or due to forces associated with lower pressure in the upper airway than outside the body. A collapse of the pharynx, larynx, upper airway or other soft tissue in the respiratory tract may thus cause the full or partial blockage, which may lead to a hypopnea or apnea event.

One method to counter collapse of the larynx is the application of constant positive airway pressure to the nostrils generally, possibly by using a CPAP machine. Application of positive airway pressure may be accomplished in the related art by placing a mask over (and sealing around) the patient's nose, and providing within the mask a pressure communicated to the pharynx, larynx, or upper airway. The pressure within the pharynx, larynx, or upper airway may be greater than the pressure outside the body, thus pneumatically splinting open the airway.

Some CPAP machines have the ability to adjust the pressure applied to the patient. In particular, some patients may have difficulty exhaling against the applied pressure, and thus some machines may implement a bi-level CPAP, with a higher pressure applied during inhalation and a lower pressure applied during exhalation. Lowering the pressure reduces the amount of pressure against which the patient must breathe during exhalation. Other CPAP machines continuously adjust the positive airway pressure applied to the mask during inhalation (even if such devices implement a bi-level system), and may be referred to in the related art as “auto titration” devices. With auto titration CPAP devices, as the patient sleeps the positive airway pressure applied is adjusted, cycling between hypoventilating (over pressuring the patient, thus causing a brain arousal) and reducing pressure to the point that the patient experiences apneas, hypopneas and/or snoring.

CPAP machines of the related art are concerned only with the pressure of the gas supplied to the mask over the patient's nose. However, gases flowing from a region of high pressure to a region of low pressure take the path of least resistance. Thus, breathable gases provided to a patient in the related art may flow only or predominantly through an open nostril or naris. Forcing or allowing gas flow to move through a single naris may cause patient discomfort, both because of the volume of the flow and because of the drying effects experienced by the naris through which the gas moves.

### SUMMARY

The problems noted above are solved in large part by a method and related system of controlling therapeutic gas provided to a patient in positive airway pressure applications. Some of the illustrative embodiments may be a method comprising supplying therapeutic gas at a first pressure to a first naris of patient during a current respiratory cycle and a subsequent respiratory cycle, selecting a second pressure based on an attribute of airflow through a second naris of the patient, and supplying therapeutic gas at the second pressure to the second naris during the current respiratory cycle.

Other illustrative embodiments are a system comprising a first blower configured to fluidly couple to a first naris of a patient, a second blower configured to fluidly couple to a second naris of a patient, and a processor electrically coupled to the first and second blowers. The processor is configured to command the first blower to a first motor speed over a previous and current respiratory cycle, and wherein the processor is configured to command the second blower a second motor speed during the current respiratory cycle, the second motor speed selected based on an attribute of airflow through the second naris.

Yet still other illustrative embodiments are a method comprising individually measuring an attribute of inhalation airflow through each naris of a patient while the patient is supplied positive airway pressure; and determining if a reduction in airflow is attributable to collapse of the patient's upper airway or a reduced airflow of one of the nares.

Other illustrative embodiments are system comprising a first blower configured to fluidly couple to a first naris of a patient, a second blower configured to fluidly couple to a second naris of the patient, and a processor. The processor is configured to command the system to supply a first pressure to the first naris over a previous and current respiratory cycle, and wherein the processor is configured to command the system to supply a second pressure to the second naris during the current respiratory cycle, the second pressure selected based on an attribute of airflow through the second naris.

Finally, other illustrative embodiments are a nasal mask comprising a nose portion configured to cover and pneumatically seal around a patient's nose (the nose portion defining an internal cavity), a supply hose fluidly coupled to the internal cavity and configured to couple to a positive airway pressure device, a first airflow attribute sensing device mechanically coupled to the nose portion configured to be within inhaled airflow of a first naris of the patient when the nose portion positioned proximate to the patient's nose, and a second airflow attribute sensing device mechanically coupled to the nose portion configured to be within inhaled airflow of a second naris of the patient when the nose portion positioned proximate to the patient's nose.

The disclosed devices and methods comprise a combination of features and advantages which enable it to overcome the deficiencies of the prior art devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the various embodiments of the invention, reference will now be made to the accompanying drawings in which:

FIG. 1 shows an illustrative system for explanation of the relationship of pressure, flow and resistance to flow;

FIG. 2 shows a system for providing positive airway pressure to a patient in accordance with at least some embodiments of the invention;

FIG. 3 shows a control system which may be used to control a positive airway pressure device in accordance with at least some embodiments of the invention;

FIG. 4 illustrates a method of controlling therapeutic gas flow through each naris in accordance with embodiments of the invention;

FIG. 5 illustrates a waveform of instantaneous measured airflow as a function of time;

FIG. 6 shows two sets of waveforms to illustrate controlling pressure on a secondary naris and an attempt to equalize airflow carried by each naris on inhalation and exhalation in accordance with embodiments of the invention;

FIG. 7 illustrates a method of equalizing airflow as between nares during exhalation in accordance with embodiments of the invention;

FIG. 8 shows a method of controlling crossover airflow in accordance with embodiments of the invention;

FIG. 9 shows a plurality of waveforms that illustrate measured airflow, and summed measured airflow;

FIG. 10 illustrates a method of discerning upper airway collapse, and a control strategy based thereon, in accordance with at least some embodiments of the invention;

FIG. 11A shows an illustrative mask that may be used in accordance with alternative embodiments of the invention; and

FIG. 11B shows a partial cut-away elevational side view of the mask of FIG. 10A.

### Notation and Nomenclature

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct connection. Thus, if a first device couples to a second device, that connection may be through a direct connection, or through an indirect connection via other devices and connections.

Further, use of the terms “pressure,” “applying a pressure,” and the like shall be in reference herein, and in the claims, to gauge pressure rather than absolute pressure.

### DETAILED DESCRIPTION OF THE PREFERRED EMBODIMENTS

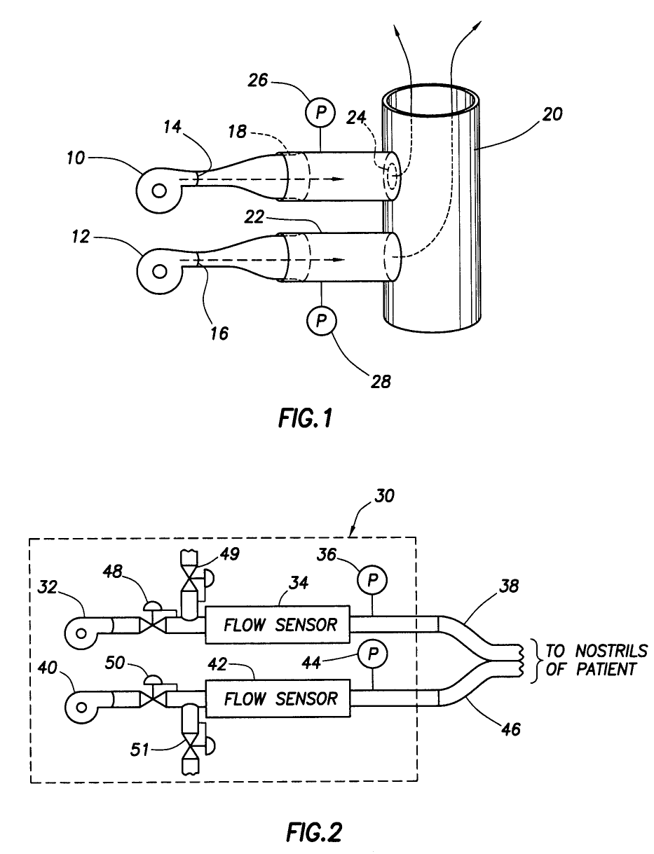

Consider, for purposes of explanation of the relationship between pressure and airflow, the system illustrated in FIG. 1. FIG. 1 illustrates a first fan or blower 10 and a second fan or blower 12. The blowers 10, 12 may be capable of providing controllable flows and/or controllable pressures on their outlet ports 14, 16 respectively. Blower 10 may have its outlet port 14 fluidly coupled to tube 18, and tube 18 may be fluidly coupled to a common chamber 20. Likewise, blower 12 may be fluidly coupled to a tube 22, and the tube 22 may likewise be coupled to the common chamber 20. In the illustration of FIG. 1, tube 18 may fluidly couple to chamber 20 through an orifice 24, and tube 22 may fluidly couple to the common chamber 20 without an orifice (or with an orifice having a flow path significantly larger than that of orifice 24). Thus, while each tube 18 and 22 is fluidly coupled to the common chamber 20, there is a restriction or resistance to airflow from the tube 18 into the chamber 20 by virtue of the orifice 24.

Further consider that each tube 18, 22 has coupled thereto a pressure transducer 26, 28 respectively. Each of the pressure transducers 26, 28 may be capable of reading a pressure within the respective tube 18, 22, and blowers 10, 12 may be operated in a pressure control mode based on pressure sensed by the pressure transducers 26, 28. While the controlled pressure within each tube may be different, for purposes of explanation consider that the pressure within each tube 18, 22 are controlled to be the same. Further consider that the common chamber 20 is at a low pressure, such as vented to atmosphere. Because of the pressure differentials between the tubes 18, 22 and the common chamber 20, there may be airflow from the tubes 18, 22 into the chamber 20 (as indicated by the arrows in FIG. 1). However, in spite of the fact that the pressures within the tubes 18, 22 are defined to be the same in this example, the airflow may be different. That is, orifice 24 provides a resistance to airflow from the tube 18 into the common chamber 20 that is not experienced by airflow moving through tube 22. Because of the restriction caused by orifice 24, the airflow through tube 18 is less than the airflow through tube 22. Thus, even for the same pressure within the tubes 18, 22, the airflow through those tubes is different under these circumstances.

Now consider that the blowers 10, 12 are operated in a flow control mode, with each blower attempting to maintain equivalent airflow regardless of required pressure. In order to maintain the desired flow, blower 10 develops a higher pressure to overcome the restriction of orifice 24 than the pressure that required of blower 12 for the same airflow. With these principles in mind, the specification now turns to a discussion of the method and related systems for providing positive airway pressure to a patient.

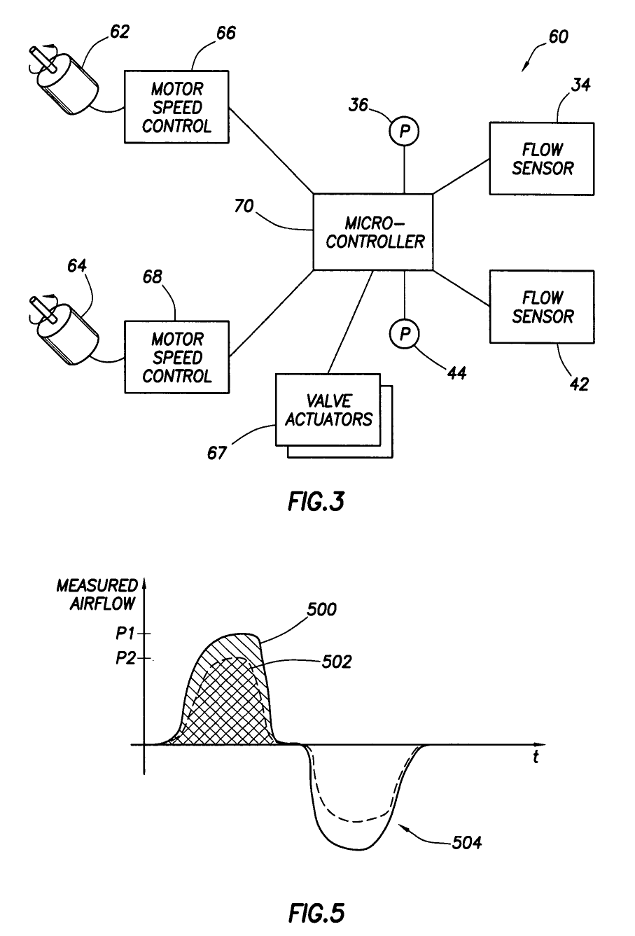

FIG. 2 illustrates a machine or device 30 for providing positive airway pressure to a patient in accordance with some embodiments of the invention. A device 30 constructed in accordance with embodiments of the invention preferably has the capability of individually controlling pressure and/or therapeutic gas flow to each nostril or naris of the patient. Thus, a first flow path comprises a blower 32 fluidly coupled to a flow sensor 34 and pressure transducer 36. Blower 32 may be any suitable device, such as a vane-type blower coupled to an electric motor. In alternative embodiments, a source of therapeutic gas, e.g. oxygen, may be used in addition to or in combination with the blower 32. Therapeutic gas pressure and flow created by the blower 32 may thus flow through the flow sensor 34 (of any suitable type) and to a first naris of a patient, possibly through tube 38. A positive airway pressure device 30 in accordance with embodiments of the invention also comprises a second blower 40 coupled to a second flow sensor 42 and second pressure transducer 44. The blower 40 may be of similar design and construction to that of blower 32. In alternative embodiments, the blower 40 may be used in combination with or replaced by a source of compressed therapeutic gas, e.g. oxygen. Therapeutic gas pressure and flow created by blower 40 may thus flow through the flow sensor 42 (of any suitable type) and to a second naris of the patient, possibly through tube 46.

In accordance with embodiments of the invention, the positive airway pressure device 30 controls pressure and/or flow to each naris of a patient individually. In some embodiments, therapeutic gas flow to the patient may be divided among the nares so as not to force any one naris to carry all the therapeutic gas flow. In order to ensure that each naris is carrying at least part of the therapeutic gas flow, the flow path for each naris may need individual pressure and/or flow control. Control of the pressure, and therefore the therapeutic gas flow, may take many forms. In some embodiments, the pressure may be controlled by selectively controlling blower speed, e.g by controlling the speed of the motor coupled to the blower. In alternative embodiments, the blowers 32, 40 may be operated at a constant speed and the pressure provided to the patient may be controlled by pressure control valves 48, 50 for the blowers 32, 40 respectively. In yet other embodiments, a combination of controlling the blower speed in a pressure control valve may be utilized. Further still, some embodiments may comprise dump valves 49 and 51, which in some embodiments vent to atmosphere during exhalation of the patient, thus reducing the pressure against which the patient exhales making exhalation less difficult.

FIG. 3 illustrates a control system 60 which may be used to control the positive airway pressure device as illustrated in FIG. 2. In particular, motors 62, 64 couple one each to blower 32, 40 (not shown in FIG. 3) respectively. The speed of the output shaft of each motor 62, 64 (and therefore the blower speed) is controlled by a motor speed control unit 66, 68 respectively. In at least some embodiments, the motors 62, 64 may be DC motors, whose speed is controlled by varying the applied DC voltages. In alternative embodiments, voltage to each of the motors 62, 64 may remain constant, but may be modulated, such as by pulse width modulation control. In yet other embodiments of the invention, the motors 62, 64 may be AC motors, and in these embodiments the motor speed control circuits 66, 68 may provide control voltages having varying voltages and frequencies to the motors so as to control motor speed. FIG. 3 also illustrates a plurality of valve actuators 67, which valve actuators couple to and control the valve position of valves 48-51.

The control system 60 also comprises a microcontroller 70 coupled to the motor speed control circuits 66, 68 and the valve actuators 67. The microcontroller 70 may be any suitable microcontroller or microprocessor programmed to provide an indication to each of the motor speed control circuits 66, 68 of a desired motor speed, and to provide an indication of valve position to the valve actuators 67. Although microprocessor control is preferred, the positive airway pressure device may be equivalently implemented with an analog control system. Setting motor speed and/or valve position for a flow circuit to a naris may be based, in some embodiments, on pressures read by the microcontroller 70 from the pressure transducers 36 and 44. In other embodiments, setting motor speed and/or valve position for a flow circuit to a naris may be based on gas flows measured by the flow sensors 34 and 42.

In accordance with some embodiments of the invention, the microcontroller 70 is provided with a doctor prescribed titration pressure, possibly by way of a dial-type input (not shown) or some other form of user interface (not shown). Based on the prescribed titration pressure, and over several minutes, the microcontroller ramps the speed control signal passed to each of the motor speed control circuits 66 and 68 and/or the valve position indications to the valve actuators 67 to achieve the prescribed titration pressure. If a naris is severely congested or otherwise blocked, however, therapeutic gas flow may move only through an open naris at the prescribed titration pressure. Moreover, throughout the night, the restriction or resistance to airflow experienced within each naris may change (e.g. as a function of congestion experienced within each naris, as a function of an amount of swelling of the soft tissue within each naris, or as a function of nasal cycle (which may be caused by brain triggered muscle contractions)). Thus, even at the prescribed titration pressure applied to each naris the patient may receive inadequate therapeutic gas.

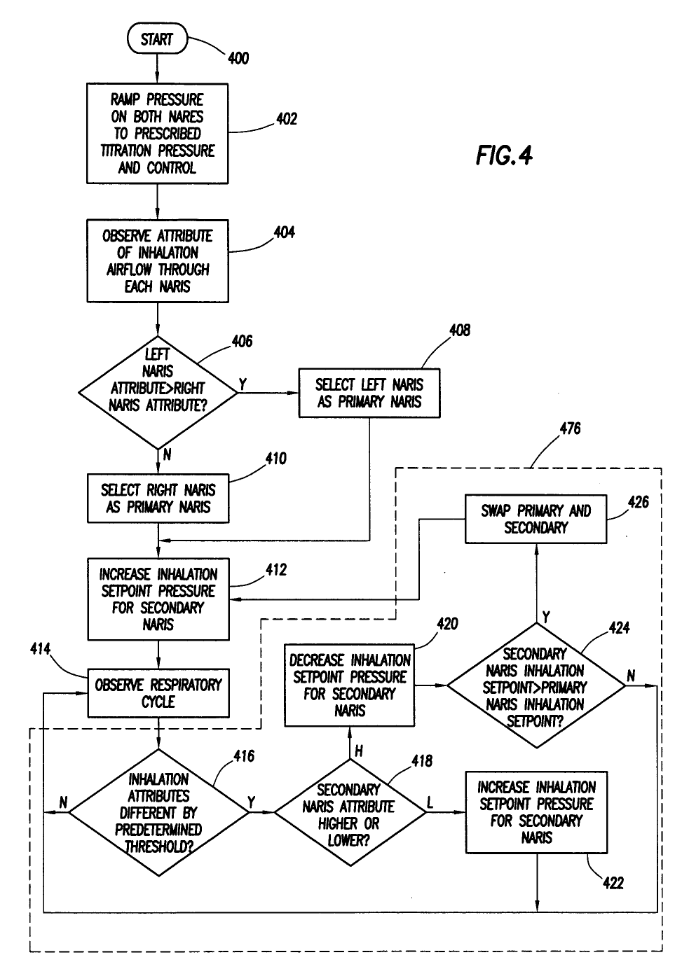

FIG. 4 illustrates a method to control therapeutic gas flow through each naris during inhalation in accordance with embodiments of the invention. In FIG. 4, or any of the figures of the specification, the order of the various illustrative steps may change, some of the illustrative steps may be combined, and others may be separated, all without departing from the scope and spirit of the invention. In particular, the process starts (block 400) and moves to ramping the applied pressure to each naris to the prescribed titration pressure, and controlling each naris at the prescribed titration pressure (block 402). The ramping may take many forms. For example, a control system 60 in accordance with embodiments of the invention may ramp from atmospheric pressure to the prescribed titration pressure over a certain period of time, e.g., ten minutes. In alternative embodiments, the pressure may be increased by a predetermined percentage on each completed respiratory cycle of the patient, and thus in these embodiments the patient's respiratory rate significantly affects the ramp time.

Regardless of the precise ramping system used, the next step is to observe an attribute of inhalation airflow through each naris of the patient (block 404). The observation may be as short as one inhalation, or may span several breaths. In the embodiments illustrated in FIG. 2, each flow path has a flow sensor, and the microcontroller (FIG. 3) may thus read the instantaneous airflow inhaled in each naris individually. In some embodiments observing the attribute of airflow through each naris involves determining a peak instantaneous airflow detected by each of the flow sensors 34, 42. In yet further alternative embodiments, the microcontroller 70 may calculate the area under the inhalation portion of the instantaneous airflow curves, which area is proportional to volume inhaled through each naris. Other therapeutic gas flow sensing mechanisms may be equivalently used (e.g., resistive thermal devices (RTDs), thermocouples, piezoelectric devices, or pressure sensors fluidly coupled to the entrance of each naris and configured to detect a drop in pressure proportional to therapeutic gas inhaled through the naris).

Next, a determination is made as to whether the left naris measured attribute of airflow is greater than the right naris measured attribute of airflow (block 406). In the illustrative case of measuring instantaneous airflow, the determination in illustrative block 406 may be a determination of which naris exhibited the highest peak instantaneous airflow. In alternative embodiments where volume is calculated using a series of instantaneous airflow measurements, the illustrative determination of block 406 may be a comparison of the total volumes carried by each naris during the inhalation. FIG. 5 illustrates a waveform of instantaneous airflow as a function of time where the airflow through each naris is individually measured. In particular, FIG. 5 shows the measured airflow signal 500 for a first naris (solid line) for an entire respiratory cycle, and also shows the measured airflow signal 502 for a second naris (dashed line) for the same respiratory cycle. In the inhalation portion (positive going airflow) of the illustrative respiratory cycle, the peak airflow for the first naris (labeled P1) is higher than the peak airflow of the second naris (labeled P2). Thus, in embodiments that use the peak instantaneous airflow as the attribute of airflow through each naris, the determination of whether the left naris attribute of airflow is greater than the right naris attribute of airflow (block 406 of FIG. 4) may be a comparison of the peak instantaneous airflows. FIG. 5 also illustrates embodiments where the attribute of airflow is the volume carried by each naris. In particular, the area under the measured airflow signal 500 (shaded by lines running upper left to lower right) for the first naris, which may be calculated by the microcontroller, is proportional to the volume carried by the first naris. Likewise, the area under the measured airflow signal 502 (shaded by lines running upper right to lower left) for the second naris is proportional to the volume carried by the second naris. Thus, in these embodiments, the determination of whether the left naris attribute of airflow is greater than the right naris attribute of airflow may be a comparison of the calculated volume carried by each naris.

For the same applied pressure, one naris carrying less airflow means the naris has a higher resistance to airflow than the open or unblocked naris. This may be caused, for example, by congestion and/or physical abnormalities of the patient. Controlling application of positive airway pressure in accordance with embodiments of the invention involves selecting a naris whose measured and/or calculated attribute of airflow is greater to be the primary naris. Thus, if the attribute of airflow of the left naris is greater, then the left naris is selected as the primary naris (block 408). If the attribute of airflow of the right naris is greater, then the right naris is selected as the primary naris (block 410). Broadly speaking, and in accordance with embodiments of the invention, control positive airway pressure with respect to the primary naris remains in a pressure control mode, while the control with respect to secondary naris shifts to a flow control mode, attempting to equalize inhalation airflow carried by each naris.

Returning to FIG. 4, inasmuch as by definition the secondary naris has less airflow than the primary naris, the next step in the illustrative process is to increase the inhalation set point pressure for the secondary naris (block 412). In some embodiments, this increase may be by a predetermined amount, e.g. 0.2 centimeters of water. In yet other embodiments, the increase in inhalation set point pressure for the secondary naris may be in proportion to the amount the airflow through the second naris was less than the primary naris, such as may be calculated in a Proportional Integral (PI) and/or a Proportional Integral Differential (PID) control loop. After making corrections to the inhalation set point pressure for the secondary naris, at least the inhalation portion of the patient's next respiratory cycle is observed (block 414). Next, a determination is made as to whether the inhalation attributes of the nares are different by a predetermined threshold (block 416). If the inhalation attributes are within the predetermined threshold (e.g. one percent of peak measured airflow and/or one percent of total volume calculated using measured airflow), then each of the patient's nares carries a substantially equal portion inhalation of the airflow and no adjustments to the secondary naris inhalation set point pressure for the next respiratory cycle are needed, and the process returns to the observation step (block 414).

If the inhalation attributes are different by more than the predetermined threshold (indicating one naris is carrying a disproportionate share of the inhalation airflow), a determination is made as to whether the secondary naris attribute of airflow is higher or lower than the primary naris attribute of airflow (block 418). If the secondary naris attribute of inhalation airflow is lower (by at least the predetermined threshold), then the inhalation set point pressure for the secondary naris is increased (block 422) in an attempt to better equalize, in the next respiratory cycle, the airflow as carried between the nares. If the secondary naris attribute of airflow is higher than the primary naris attribute of airflow, the inhalation set point pressure for the secondary naris is decreased (block 420). Having the secondary naris airflow higher than the primary naris is indicative of either an overcorrection of inhalation set point pressure for the secondary naris, or that the blockage of the secondary naris that initially caused the naris to carry less airflow has subsided (congestion has gone away, or the patient has changed physical position of the head, thus, physiologically, opening the naris).

Throughout the course of sleeping overnight, or any situation where application of positive airway pressure is desirable, the ability of each naris to carry airflow may change with time. For example, a patient may experience a full or partial blockage of one naris due to mucous accumulations, and because of head position and gravity the mucous may migrate to and fully or partially block the second naris. Likewise, some patients have physical attributes which affect airflow through each naris differently depending, for example, on head position. Thus, it is possible that a naris initially selected as the primary naris develops a resistance to airflow. As the initially selected primary naris develops resistance to airflow and therefore a drop in airflow, the illustrative control system 60 likewise decreases the inhalation set point pressure for the secondary naris to track the decrease in airflow of the primary naris. However, when the inhalation set point pressure of the secondary naris falls to the prescribed titration pressure, the primary and secondary naris designations are swapped. That is, if the left naris was previously selected as the primary naris but has developed a resistance to airflow, when the inhalation set point pressure for the right naris falls to the prescribed titration pressure, then the right naris is selected as the primary naris (and placed on pressure control) and the left naris is designated at the secondary naris whose airflow is thus controlled to match the new primary naris. Thus, if the secondary naris inhalation set point pressure is decreased (block 420), then a determination is made as to whether the secondary naris inhalation set point pressure is the same or less than the primary naris inhalation set point pressure (block 424) (which is, in some embodiments, the prescribed titration pressure). If so, then the primary and secondary designations of the nares are swapped (block 426), and the next step is to increase the inhalation set point pressure for the new secondary naris (again block 412). If, on the other hand, the secondary naris inhalation set point is greater than the primary naris inhalation set point (again block 424) (preferably the prescribed titration pressure), or there was an increase in secondary naris inhalation pressure (block 422), then the illustrative control system 60 begins the process anew by observing at least a portion of a respiratory cycle (again block 414).

Summarizing before continuing, a control system 60 in accordance with embodiments of the invention selects a primary naris (the selection based on which naris carries the most inhalation airflow when both nares are provided the same pressure), controls the primary naris applied pressure to be the prescribed titration pressure, and controls inhalation airflow of the secondary naris (somewhat without regard to pressure required to achieve the airflow) in an attempt to evenly distribute the airflow carried by each naris during inhalation.

Referring again to FIG. 5, the illustrative method discussed to this point (FIG. 4) concerned itself only with attempting to evenly distribute the airflow carried by each naris during inhalation. However, the restrictions that limit airflow during inhalation may also limit airflow during exhalation. Moreover, for a variety of reason (e.g., anatomical anomalies), the naris that exhibits less resistance to airflow during inhalation may exhibit greater resistance to airflow during exhalation. The following discussion assumes that the naris exhibiting greater inhalation airflow will also exhibit greater exhalation airflow so as not to unduly complicate the discussion, but the alternate situation is also contemplated. Still referring to FIG. 5, the exhalation portion 504 of the illustrative instantaneous measured airflow of FIG. 5 also illustrates that in some cases the secondary naris may carry less airflow during exhalation than the first naris. In addition to attempting to evenly distribute airflow carried by each naris during inhalation, some embodiments also attempt to evenly distribute airflow carried by each naris during exhalation.

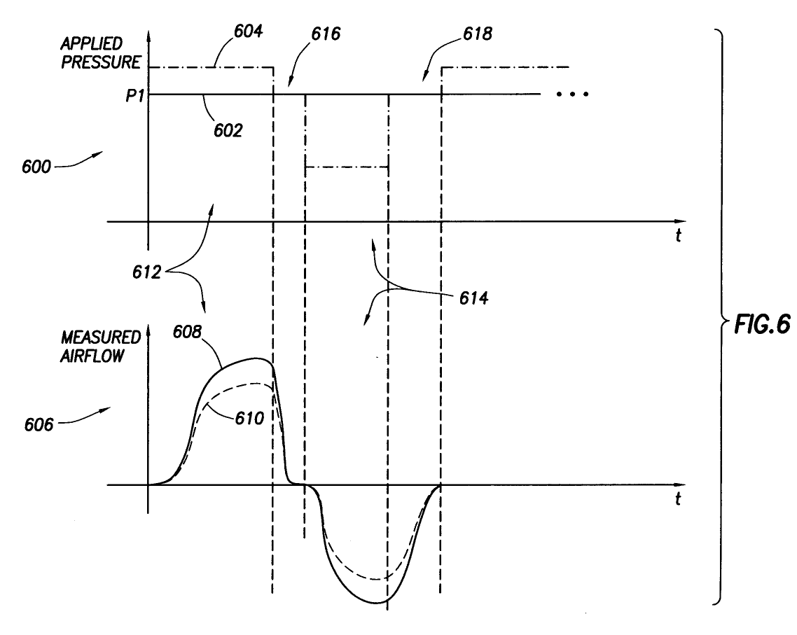

FIG. 6 shows two sets of waveforms to better illustrate controlling pressure on the secondary naris in an attempt to equalize airflow carried by each naris both on inhalation and exhalation. In particular, waveforms 600 show positive airway pressure as a function of time applied to each naris. Signal 602 is illustrative of pressure applied to the primary naris, and signal 604 (dash-dot-dash) is illustrative of pressure applied to the secondary naris. Waveforms 606 show measured airflow signals as a function of time, plotted on the same vertical axes as the applied pressure waveforms 600, but on a different, though corresponding, time axis. In particular, signal 608 is illustrative of the instantaneous airflow of primary naris, and signal 610 (dashed line) is illustrative of measured airflow for a secondary naris. Notice how the pressure applied to the secondary naris (signal 604) is higher than the pressure applied to the primary naris (signal 602) during inhalation (region 612). This relationship is illustrative of the pressure control methodology discussed with respect to FIG. 4. However, and in accordance with alternative embodiments of the invention, in an attempt to allow the secondary naris to carry approximately the same airflow during exhalation as the primary naris, the pressure applied during exhalation (region 614) drops below that applied to the primary naris (there is a period of time when the pressures are the same, and this is discussed more fully below). Although not specifically shown, pressure applied to the primary naris too could be lowered during exhalation, and the lowered pressures need not necessarily be the same. Stated otherwise, there could still be some attempt to equalize exhalation airflow as between the nares even when both nares are provided a lower pressure on exhalation.

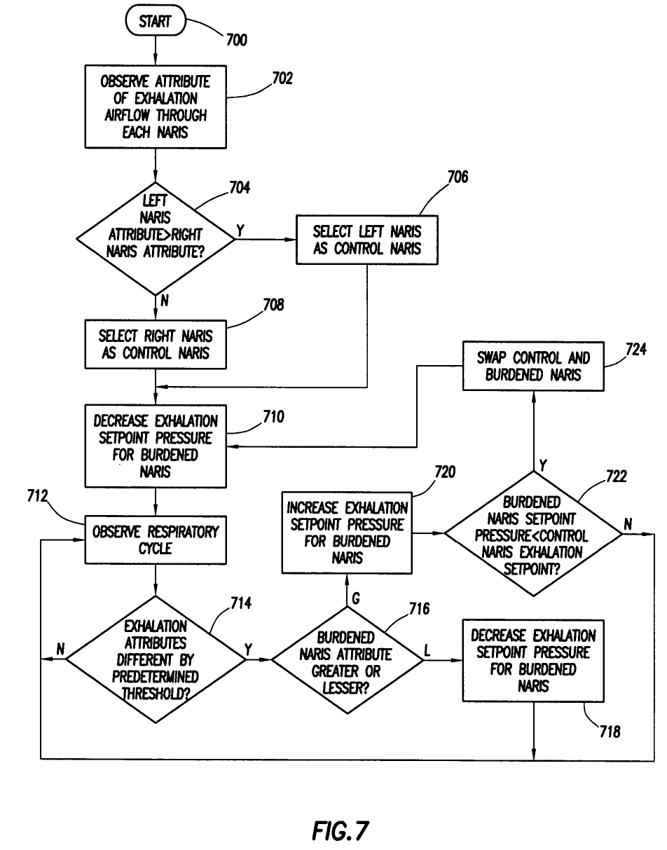

FIG. 7 illustrates a method to control exhalation airflow through each naris in accordance with embodiments of the invention. In particular, the process starts (block 700) and moves to observing an attribute of airflow through each naris of the patient (block 702), which may be coextensive with the observation as block 404 of FIG. 4. In some embodiments observing the attribute of airflow through each naris involves determining a peak instantaneous exhalation airflow detected by each of the flow sensors 34, 42. In alternative embodiments, the microcontroller 70 may calculate the area under the exhalation portion of the instantaneous airflow curves, which area is proportional to volume exhaled through each naris. Other therapeutic gas flow sensing mechanisms may be equivalently used.

Next, a determination is made as to whether the left naris measured attribute of exhalation airflow is greater than the right naris measured attribute of airflow (block 704). In the illustrative case of measuring instantaneous exhalation airflow, the determination in illustrative block 704 may be a determination of which naris exhibited the lowest peak (largest negative value if no flow is zero and exhalation airflows are negative relative to positive going inhalation airflows) instantaneous airflow. In alternative embodiments where volume is calculated using a series of instantaneous airflow measurements, the illustrative determination of block 704 may be a comparison of the total volumes carried by each naris during the exhalation.

For the same applied pressure during exhalation, one naris carrying less airflow means the naris has a higher resistance to exhalation airflow than the open or unblocked naris. This may be caused, for example, by congestion and/or physical abnormalities of the patient. Controlling application of positive airway pressure to attempt to equalize airflow during exhalation involves selecting a naris whose attribute is greater (e.g., more negative if using peak values of airflow with no flow taking on a zero value, or greater exhalation volume) to be the control naris. In most cases the control naris for exhalation purposes and the primary naris for inhalation purposes will be the same, but this is not necessary always the case. Thus, if the attribute of airflow of the left naris is greater, then the left naris is selected as the control naris (block 706). If the attribute of airflow of the right naris is greater, then the right naris is selected as the control naris (block 708). The naris not selected as the control naris is termed the “burdened naris.” Broadly speaking, and in accordance with some embodiments of the invention, control of positive airway pressure applied during exhalation with respect to the control naris remains in a pressure control mode, while the control with respect to burdened naris shifts to a flow control mode, attempting to equalize exhalation inhalation airflow carried by each naris.

Returning to FIG. 7, inasmuch as by definition the burdened naris has less exhalation airflow than the control naris, the next step in the illustrative process is to decrease the exhalation set point pressure for the burdened naris (block 710) (decreasing the pressure applied during exhalation makes it easier to exhale through the burdened naris, thus increasing exhalation airflow). In some embodiments, this decrease may be by a predetermined amount, e.g. 0.2 centimeters of water. In yet other embodiments, the decrease in exhalation set point pressure for the burdened naris may be in proportion to the amount the attribute of airflow through the burdened naris was less than the control naris, such as may be calculated in a PI and/or PID control loop. After making corrections to the exhalation set point pressure for the burdened naris, at least the exhalation portion of the patient's next respiratory cycle is observed (block 712). Next, a determination is made as to whether the exhalation attributes of the nares are different by a predetermined threshold (block 714). If the exhalation attributes are within the predetermined threshold (e.g. one percent of peak measured airflow and/or one percent of total volume calculated using measured airflow), then each of the patient's nares carries a substantially equal portion of the exhalation airflow and no adjustments to the burdened naris exhalation set point pressure for the next respiratory cycle are needed, and the process returns to the observation step (block 712).

If the exhalation attributes are different by more than the predetermined threshold (indicating one naris is carrying a disproportionate share of the exhalation airflow), a determination is made as to whether the burdened naris attribute of airflow is greater or lesser than the control naris attribute of airflow (block 716). If the burdened naris attribute of exhalation airflow is lower by at least the predetermined threshold (that is, e.g., having a peak airflow that is less negative than the control naris, or a total volume that is less than the control naris), then the inhalation set point pressure for the burdened naris is decreased (block 718) in an attempt to better equalize, in the next respiratory cycle, the airflow as carried between the nares. If the burdened naris attribute of airflow is greater than the control naris attribute of airflow (that is, e.g., having a peak airflow that is more negative than the control naris, or a total volume that is greater than the control naris), the inhalation set point pressure for the burdened naris is increased (block 720) (raising the pressure applied during exhalation makes it more difficult to breath out the burdened naris). Having the burdened naris exhalation airflow greater than the control naris is indicative of either an overcorrection of exhalation set point pressure for the burdened naris, or that the blockage of the burdened naris that initially caused the naris to carry less airflow has subsided (congestion has gone away, or the patient has changed physical position of the head, thus, physiologically, opening the naris).

When the exhalation set point pressure of the burdened naris rises to a preselected exhalation set point pressure (discussed more below), the control and burdened naris designations are swapped. That is, if the left naris was previously selected as the control naris but has developed a resistance to exhalation airflow, when the exhalation set point pressure for the right naris rises to the preselected exhalation set point pressure, then the right naris is selected as the control naris (and placed on pressure control) and the left naris is designated at the burdened naris whose airflow is thus controlled to match the new control naris. Thus, if the burdened naris exhalation set point pressure is increased (block 720), then a determination is made as to whether the burdened naris exhalation set point pressure is the same or greater than the control naris exhalation set point pressure (block 722). If so, then the control and burdened designations of the nares are swapped (block 724), and the next step is to decrease the exhalation set point pressure for the new burdened naris (again block 710). If, on the other hand, the burdened naris exhalation set point is less than the control naris exhalation set point (again block 722), or there was a decrease in burdened naris exhalation pressure (block 720), then the illustrative control system 60 begins the process anew by observing at least a portion of a respiratory cycle (again block 712).

The preselected exhalation set point pressure, to which the pressure of the control naris is controlled, may take several values. In some embodiments, the preselected exhalation set point pressure is the same as the prescribed titration pressure. In other embodiments, the preselected exhalation set point pressure is lower than the prescribed titration pressure, and thus a device 30 in accordance with these embodiments may lower the pressure applied to the control naris during exhalation, and yet also attempt to equalize exhalation airflow by lower further still the pressure applied during exhalation to the burdened naris. One or both of the pressures applied to the nares during exhalation could be below atmospheric.

Returning to FIG. 6, in accordance with embodiments of the invention, the higher pressure applied to the secondary naris during inhalation is preferably brought back to the prescribed titration pressure after reaching the peak airflow, as illustrated in region 616 of the waveforms 600 (FIG. 6 shows an idealized case, with pressure change being instantaneous; however, pressure may change over time, and in some embodiments may decay at a rate proportional to the decay of inhalation airflow). Similarly for embodiments that apply different pressures on exhalation, the applied pressure on the burdened naris is preferable brought back to the preselected exhalation set point pressure just after the peak exhalation airflow (region 618 of waveforms 600) (again, FIG. 6 shows an idealized case). An explanation for this control philosophy requires a brief diversion to FIG. 1. In particular, consider the situation where the common chamber 20 is sealed, rather than vented to atmosphere, which is illustrative of the situations where a patient is neither inhaling nor exhaling (as well as times of small respiration volume at the end of the inhalation cycle and beginning of the exhalation cycle, and likewise at the transition from exhalation to inhalation). Further consider that blower 10 applies a greater pressure to tube 18 than blower 12 to tube 22. Under these assumption, and unless one naris is blocked, there will be a “crossover” flow from tube 18 to tube 22, with the amount of flow proportional to the difference in pressure and the resistance to airflow of orifice 24. The inventors herein have found that crossover flow occurs in positive airway pressure applications where the pressures to the nares are independently controlled. In particular, if the pressures applied to the nares are different at times of small respiration volume, there tends to be a crossover flow from the naris being supplied the higher pressure to the naris being supplied the lower pressure. Returning to FIG. 6, regions 616 and 618 thus illustrate a control philosophy to reduce the crossover airflow, the control philosophy being to control the pressure applied to the nares to be the approximately the same during times of small respiratory volume.

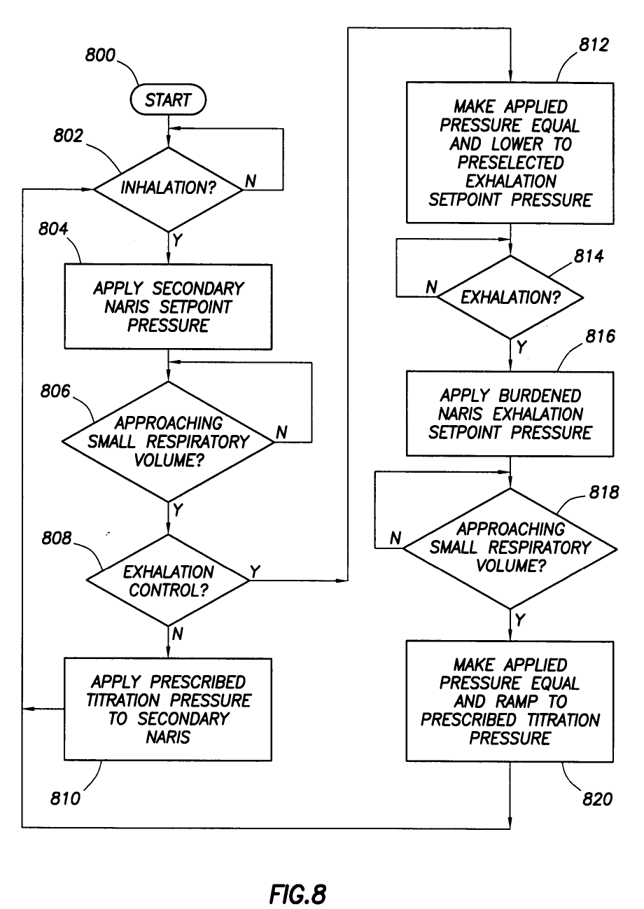

FIG. 8 illustrates a method in accordance with embodiments of the invention to address the crossover airflow phenomenon. The illustrative methods of FIGS. 4 and 7 (if implemented) preferably run substantially simultaneously with the method of FIG. 8, with FIG. 8 applying the pressures as set by FIGS. 4 and 7. In particular, the method starts (block 800) and moves to sensing an inhalation (block 802). Sensing an inhalation may take many forms. In some embodiments, the inhalation is sensed when the patient actually begins to inspire. In alternative embodiments, the inhalation can be predicted by observing the state of the exhalation airflow. When an inhalation is sensed, the secondary naris set point pressure is applied (block 804), which pressure will be higher than the prescribed titration pressure in an attempt to equalize inhalation airflow as between the nares.

Thereafter, the illustrative method makes a determination of whether the inhalation is approaching a small respiratory volume (block 806). This illustrative step may take many forms. In some embodiments, detecting instantaneous inhalation airflow maxima is indicative of an approaching small respiratory volume. Alternative embodiments may use the slope of the instantaneous airflow curve, or reaching a predetermined airflow, as indications of an approaching small respiratory volume. Once the small respiratory volume is approaching, a determination is made as to whether the particular system implements exhalation control (block 808). If no exhalation control is implemented (e.g., the patient may not need exhalation relief), then the illustrative system applies the prescribed titration pressure to the secondary naris (block 810) (region 616 of FIG. 6), and keeping in mind the primary naris is controlled to the prescribed titration pressure. Thereafter, the system waits again for an inhalation (block 802).

If the system implements exhalation control (again block 808), then the pressures applied to each naris are made equal and lowered to the preselected exhalation set point pressure (block 812). The preselected exhalation set point pressure could be the same as the prescribed titration pressure or below, including below atmospheric pressure (ventilator-type applications). When the exhalation is sensed (block 814), the illustrative method then applies the burdened naris exhalation set point pressure to the burdened naris (block 816). Sensing the exhalation could be anything from predictive (predicted based on tapering of inhalation airflow) to reactive (sensed only when actual exhalation airflow is sensed). The waveforms 600 of FIG. 6 illustrate the situation where exhalation control is implemented with the preselected exhalation set point pressure set equal to the prescribed titration pressure. The exhalation pressures are applied until a determination is made that there is an approaching small respiratory volume (block 818). As with respect to the inhalation, sensing an approaching small respiratory volume may take many forms, e.g., sensing exhalation airflow maxima, slope determinations, reaching predetermined thresholds, and the like. When the small respiratory exhalation air volume is approaching, the illustrative method makes the pressures applied to the nares equal (to reduce cross-over flow), and ramps to the prescribed titration pressure (block 820). Thereafter, the process begins anew by looking for an inhalation (block 802).

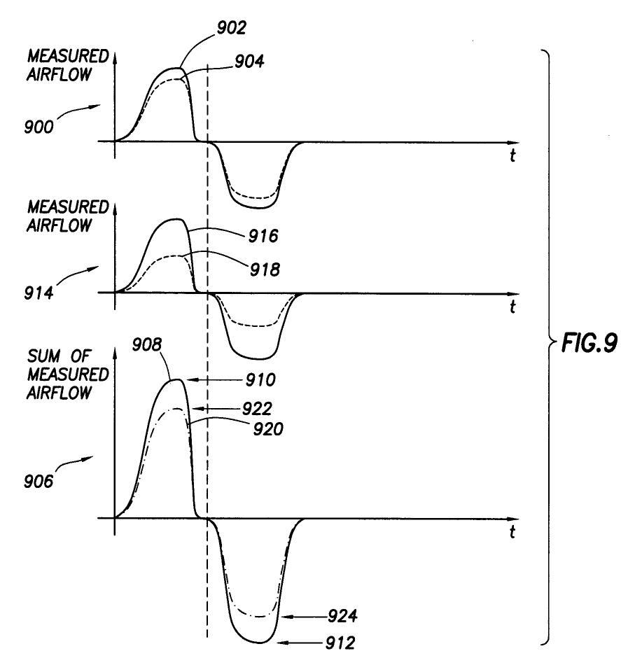

As discussed in the Background section, some related art “auto titration” CPAP machines adjust the pressure applied to a mask over the patient's nose in an attempt to lower the applied pressure (thus increasing patient comfort) at times when a lower pressure will suffice. Further, the auto titration CPAP machines raise applied pressure when the patient experiences apneas, hypopneas, snoring and/or upper airway collapse. However, the inventors of the present specification have found that cyclic congestion and clearing of a naris can be falsely interpreted by related art auto titration CPAP machines as an upper airway collapse, which in turn precipitates an increase in the applied pressure by the auto-titration machines. Referring to FIG. 9, waveforms 900 illustrate an instantaneous airflow signal 902 for a first naris and a signal 904 for a second naris (dashed line) during a single respiratory cycle. Waveforms 906 illustrate, and in particular signal 908, a point-for-point summation of the instantaneous airflows signals 902 and 904. Notice how the illustrative signal 908 has an inhalation peak 910, and a corresponding exhalation peak 912. Waveforms 914 illustrates an instantaneous airflow signal for a first naris (signal 916) which carries the same airflow as the first naris of signal 902; however, instantaneous airflow signal 918 for a second naris shows a significant drop in airflow with respect to the second naris signal 904. In this illustrative situation, the fact that the first naris (signals 902 and 916) has the same airflow is indicative of a lack of full or partial upper airway collapse. The drop in airflow for the illustrative second naris (illustrated by signal 918) shows that the resistance to airflow exists primarily only in the second naris. Signal 920 is a point-for-point summation of the signals 916 and 918 over the complete respiratory cycle. Notice how the positive peak 922 is significantly less than the positive peak 910 of signal 908. Notice also how the negative peak 924 of signal 920 shows less carried airflow with respect to peak 912 of signal 908. Thus, related art auto titration CPAP devices see the reduction in airflow (differences between peaks 910 and 922, and/or negative peaks 912 and 924) as an upper airway collapse, and increase applied pressure; however, signal 920 is built under the assumption that the first naris carries the same airflow in each case, and it is only a blockage within the second naris that caused the overall drop in carried airflow. Thus, the related art reaction of increasing applied pressure to both nares is not needed.

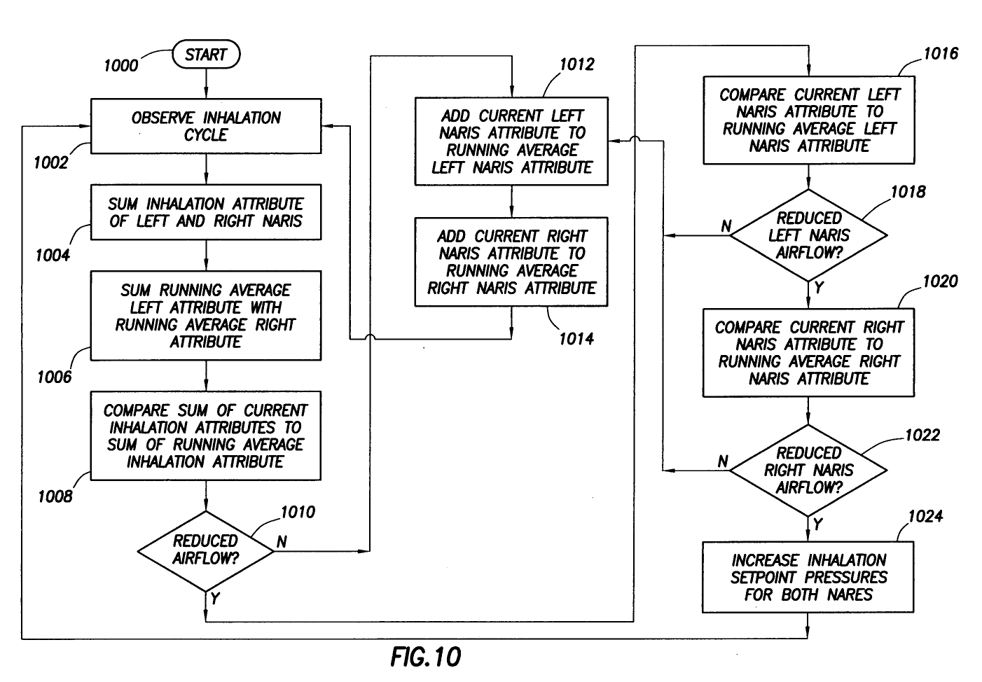

FIG. 10 is a flow diagram of a method of discerning upper airway collapse from a drop of an inhaled airflow caused by blockage of a single naris, and a control strategy based thereon in accordance with at least some embodiments of the invention. In particular, the process starts (block 1000), and proceeds to observation of an inhalation cycle (block 1002) (which may be a portion of the observation as illustrated in FIG. 4, block 414). After observing an inhalation cycle, the illustrative method sums the inhalation attribute of the left and right naris (block 1004). For example, if the inhalation attribute is the peak instantaneous airflow, then illustrative step 1004 sums the peak instantaneous airflow as between the left and the right nares. Likewise, if the inhalation attribute is the volume calculated using the measured airflow as a function of time, then illustrative step 1004 involves summing the volumes calculated. Next, illustrative method sums a running average left attribute with the running average right attribute (block 1006) (calculation of these running averages is discussed more fully below), and compares the sum of the current inhalation attributes to the sum of the running average inhalation attributes (block 1008). Thereafter, a determination is made as to whether there is reduced airflow as between the current inhalation and the running average inhalation (block 1010). If the comparison does not indicate a reduced airflow, or possibly the current inhalation airflow is within a predetermined threshold of the running average, then the current left naris attribute of airflow is added to the running average left naris attribute (block 1012), and likewise the current right naris attribute of airflow is added to the running average right naris attribute (block 1014). In at least some embodiments, the running average for the left and right naris attribute of airflow is the running average for the last 2 to 10 minutes, with a 5-minute running average preferred. Thereafter, the process starts anew by observing the inhalation cycle (again block 1002).

If the comparison of the sum of the current inhalation attributes of airflow to the sum of the running average inhalation attributes shows a reduction in the airflow (again block 1010), then a comparison is made as between the current left naris attribute of airflow and the running average left naris attribute of airflow (block 1016). If a reduction in airflow is noted in the left naris (block 1018), then a comparison is made between the current right naris attribute of airflow and the running average right naris attribute of airflow (block 1020). Embodiments of the illustrative method then determine whether there is reduced right naris airflow (block 1022). Thus, if both the right and left naris have reduced airflow, this is indicative of a collapse of at least the patient's upper airway, which triggers an increase in the inhalation set point pressures for both nares (block 1024), and the process begins anew by observation of the inhalation cycle (block 1002).

Still referring to FIG. 10, and returning to illustrative blocks 1018 and 1022. If no reduction in airflow is noted in one naris (blocks 1018 or 1022), this is indicative that the reduction in airflow that triggered the specific comparisons is not caused by an upper airway collapse, and instead is caused by a congestion and/or blockage of a single naris. Thus, in the case where the reduction in airflow is only within a single naris, the current left naris attribute of airflow is added to the running average left naris attribute of airflow (block 1012), the current right naris attribute of airflow is added to the running average right naris attribute of airflow (block 1014), and the process begins anew by observation of the inhalation cycle (again block 1002). If the illustrative method of FIG. 4 runs simultaneously or substantially simultaneously with the method of FIG. 10, this illustrative method increases the inhalation set point for the naris experiencing a drop in airflow in an attempt to equalize airflow carried by the naris.

Thus, by separately monitoring the airflow of each naris, and likewise being able to independently control the pressure applied to each naris, an illustrative control system 60 in accordance with embodiments of the invention is able to selectively increase pressure in both nares to compensate for upper airway collapse, or adjust the inhalation set point pressure for a single naris to compensate for congestion and/or blockage present within that naris. Alternative embodiments of the invention, however, sense individual attributes of airflow through each naris, yet provide only a single controllable pressure to the nares. In particular, in these alternative embodiments of the invention, an attribute of the airflow through each naris is independently measured. By comparing current individual naris attributes of airflow to running average individual naris attributes of airflow, these embodiments determine whether a reduction in airflow is attributable to an upper airway collapse, or congestion and/or blockage in a single naris.

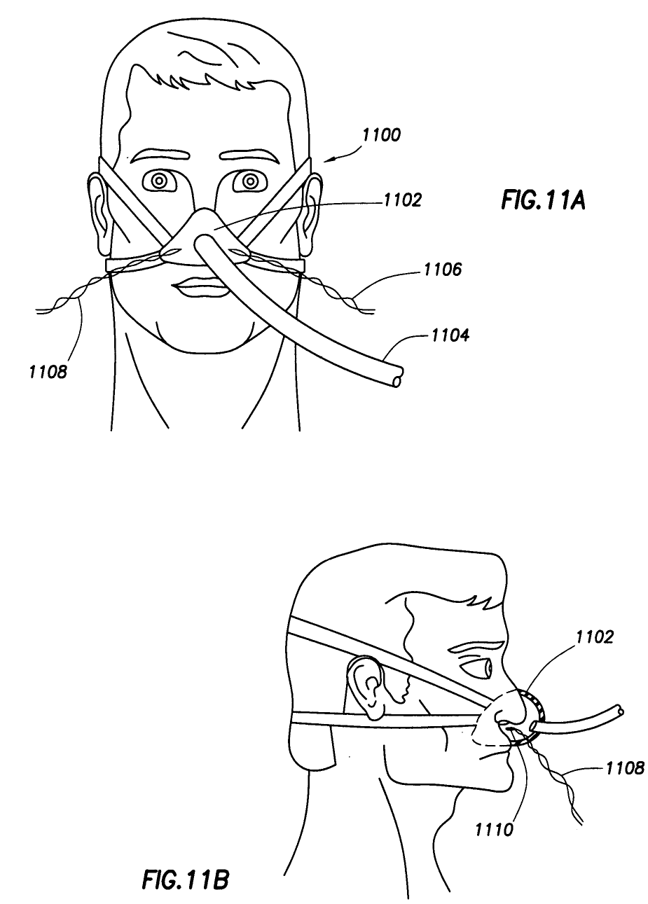

FIG. 11A shows an illustrative mask 1100 that may used in accordance with these alternative embodiments of the invention. In particular, the mask comprises a nose piece 1102, which covers the patient's nose (and therefore both nostrils generally), and couples to a single plenum 1104 which in turn fluidly couples to a source of therapeutic gas at increased pressure. The plenum 1104 may couple, for example, to a single flow path of the illustrative control system 30 (of FIG. 2). The mask 1100 also comprises a first connection 1106 and a second connection 1108, each of which individually couples to an attribute measuring device within the nose portion 1102. FIG. 11B shows a partial cut-away elevational side view of the mask to illustrate placement of the attribute measuring devices. For example, the connection 1108 may couple to a temperature sensing device 1110 placed in operational relationship to a naris. The measuring device 1110 may thus measure an attribute of airflow for the particular naris. The measuring device 1110, however, may be any suitable device and/or system, such as a thermocouple, RTD, piezoelectric device, or any other device now in existence or after developed that senses increased temperature associated with exhalation. In yet further alternative embodiments, the attribute sensing device 1110 may sense a pressure proximate to the opening of the naris, with a magnitude of reduced pressure indicative of inhaled airflow into the naris, and with a magnitude of increased pressure indicative of exhaled airflow. The actual pressure sensor, which may be located outside the nose portion 1102, yet having a tube fluidly connecting the pressure sensor and terminating proximate to the opening of the naris, senses a baseline pressure similar to that applied as a titration pressure, and with the breathing waveform “riding” the sensed baseline pressure. The breathing waveform, in its analog or digital form, may be separated from the sensed baseline pressure by an analog or digital high pass filter respectively. In yet still other alternative embodiments, each attribute sensing device 1110 may take the form of a pressure sensor inside the mask, with first and second connections 1106, 1108 coupled one each to the pressure sensors inside the mask. Further still, each attribute sensing device 1110 may be a mass flow sensor within the mask, with first and second connections 1106, 1108 coupled one each to the mass flow sensors inside the mask.

Alternative embodiments of the invention may thus determine whether a reduction in airflow is an upper airway collapse (airflow in both nares drops proportionately) or whether the reduction in airflow is caused by congestion and/or blockage in a single naris. In this latter case, while a reduction in airflow may be noted, increasing the positive airway pressure to both nares would have the detrimental effect of further increasing the airflow through the open naris, and possibly over pressuring and thus hypoventilating the patient.

Dividing the airflow substantially evenly between the nares may be more comfortable for the patient. A device that is capable of adjusting its pressure to ensure airflow may more accurately provide that airflow in spite of the fact that the resistance to flow through a naris may change significantly over the course of a night. Further, dividing the therapeutic gas flow among the nares substantially evenly may reduce discomfort associated with drying of the nasal cavities by the airflow through the nares (in comparison to forcing the airflow through a single naris).

From the description provided herein, those skilled in the art are readily able to combine software created as described with appropriate general purpose or a special purpose computer hardware to create a computer system and/or computer subcomponents embodying aspects of the invention, to create a computer system and/or computer subcomponents for carrying out the method embodiments of the invention, and/or to create a computer-readable medium storing a software program to implement method aspects of the various embodiments. Moreover, the embodiments of the illustrative methods could be implemented together in a single program (with various subroutines), or split up into two or more programs executed on the processor.

The above discussion is meant to be illustrative of the principles and various embodiments of the present invention. Numerous variations and modifications will become apparent to those skilled in the art once the above disclosure is fully appreciated. For example, the various embodiments discuss setting set point pressures based on a current attribution of respiration to make flow corrections in a subsequent respiration; however, pressure set points may be adjusted to make flow corrections within the same inhalation or exhalation. It is intended that the following claims be interpreted to embrace all such variations and modifications.

What is claimed is:

1. A method comprising:
supplying therapeutic gas at a first pressure to a first naris of patient during a current respiratory cycle and a subsequent respiratory cycle;
selecting a second pressure based on an attribute of airflow through a second naris of the patient; and
supplying therapeutic gas at the second pressure to the second naris during the current respiratory cycle.

2. The method as defined in claim 1 wherein selecting further comprises selecting the second pressure to make more equal airflow carried as between the first naris and the second naris than if the first and second pressures were the same.

3. The method as defined in claim 2 wherein selecting further comprises selecting the second pressure to make airflow during the current respiratory cycle as between the first naris and second naris substantially equal.

4. The method as defined in claim 1 wherein selecting further comprise selecting based on the attribute of a previous respiratory cycle.

5. The method as defined in claim 1 wherein selecting further comprise selecting based on the attribute of the current respiratory cycle.

6. The method as defined in claim 1 wherein selecting further comprises selecting based on a peak instantaneous airflow of the second naris.

7. The method as defined in claim 1 wherein selecting further comprises selecting based on a total volume carried by the second naris.

8. The method as defined in claim 1 further comprising:
wherein supplying therapeutic gas at the first pressure further comprises supplying the therapeutic gas at the first pressure during an inhalation of the current respiratory cycle; and
wherein supplying therapeutic gas at the second pressure further comprises supplying the therapeutic gas at the second pressure during the inhalation.

9. The method as defined in claim 8 further comprising:
supplying therapeutic gas at a third pressure to the first naris during an exhalation of the current respiratory cycle; and
selecting a fourth pressure based on an attribute of airflow through a second naris of the patient, the selecting to make more equal airflow carried as between the first naris and the second naris than if the third and fourth pressures were the same; and
supplying therapeutic gas at the fourth pressure to the second naris during the exhalation.

10. The method as defined in claim 9 wherein the first and third pressures are the same.

11. The method as defined in claim 8 further comprising discerning if a reduction in total airflow of the first naris and second naris is attributable to collapse of the patient's upper airway or a reduced airflow of one of the nares.

12. The method as defined in claim 1 further comprising:
wherein supplying therapeutic gas at the first pressure further comprises supplying the therapeutic gas at the first pressure during exhalation; and
wherein supplying therapeutic gas at the second pressure further comprises supplying the therapeutic gas at the second pressure during the exhalation.

13. The method as defined in claim 1 further comprising equalizing the first and second pressure during periods of small or no respiratory volume.

14. A system comprising:
a first blower configured to fluidly couple to a first naris of a patient;
a second blower configured to fluidly couple to a second naris of the patient;
a processor electrically coupled to the first and second blowers;
wherein the processor is configured to command the first blower to a first motor speed over a previous and current respiratory cycle, and wherein the processor is configured to command the second blower a second motor speed during the current respiratory cycle, the second motor speed selected based on an attribute of airflow through the second naris.

15. The system as defined in claim 14 wherein the processor is configured to command the second blower to the second motor speed selected to make more equal airflow carried as between the first naris and the second naris during the current respiratory cycle than if the first and second motor speeds were the same.

16. The system as defined in claim 15 wherein the processor is configured to command the second blower to the second motor speed to make airflow during the current respiratory cycle as between the first naris and second naris substantially equal.

17. The system as defined in claim 14 wherein the processor is configured to command the second blower to the second motor speed selected based on the attribute of the previous respiratory cycle.

18. The system as defined in claim 14 wherein the processor is configured to command the second blower to the second motor speed selected based on the attribute of the current respiratory cycle.

19. The system as defined in claim 14 wherein the processor is configured to command the first blower to the first motor speed during an inhalation of the current respiratory cycle, and wherein the processor is configured to command the second blower to the second motor speed during the inhalation.

20. The system as defined in claim 19 further comprising wherein the processor is configured to command the first blower to a third motor speed during an exhalation of the current respiratory cycle, and wherein the processor is configured to command the second blower to a fourth motor speed during the exhalation, the fourth motor speed selected to make more equal airflow carried as between the first naris and the second naris than if the third and fourth motor speeds were the same.

21. The system as defined in claim 20 wherein the first and third motor speeds are the same.

22. The system as defined in claim 19 wherein the processor is further configured to discern if a reduction in total airflow of the first naris and second naris is attributable to collapse of the patient's upper airway or a reduced airflow of one of the nares.

23. The system as defined in claim 14 wherein the processor is configured to command the first blower to the first motor speed during an exhalation of the current respiratory cycle, and wherein the processor is configured to command the second blower to the second motor speed during the exhalation.

24. The system as defined in claim 14 wherein the processor is further configured to equalize the first and second motor speeds during periods of small or no respiratory volume.

25. A method comprising:
individually measuring an attribute of inhalation airflow through each naris of a patient while the patient is supplied positive airway pressure; and
determining if a reduction in airflow is attributable to collapse of the patient's upper airway or a reduced airflow of one of the nares.

26. The method as defined in claim 25 further comprising increasing positive airway pressure to both nares only if the determining step reveals a collapse of the patient's upper airway.

27. The method as defined in claim 25 wherein individually measuring further comprises measuring instantaneous airflow, and determining a peak instantaneous airflow during the inhalation.

28. The method as defined in claim 25 wherein individually measuring further comprises measuring instantaneous airflow, and calculating volume inhaled using the instantaneous airflow measured during the inhalation.

29. The method as defined in claim 25 wherein individually measuring further comprises measuring a temperature associated with airflow.

30. The method as defined in claim 25 determining further comprises:
comparing an attribute of inhalation of a current breath of a first naris to a running average of the attribute of inhalation of the first naris;
comparing an attribute of inhalation of a current breath of the second naris to a running average of the attribute of inhalation of the second naris; and
comparing a sum of the attributes of inhalation of the current breath for the nares to a running average sum of the attributes of inhalation of the nares.

31. The method as defined in claim 25 wherein determining further comprises determining the presence of an upper airway collapse by a reduction in airflow of both nares.

32. The method as defined in claim 25 wherein determining further comprises determining the absence of an upper airway collapse by a reduction in airflow substantially only one naris.

33. A system comprising:
a first blower configured to fluidly couple to a first naris of a patient;
a second blower configured to fluidly couple to a second naris of the patient;
a processor;
wherein the processor is configured to command the system to supply a first pressure to the first naris over a previous and current respiratory cycle, and wherein the processor is configured to command the system to supply a second pressure to the second naris during the current respiratory cycle, the second pressure selected based on an attribute of airflow through the second naris.

34. The system as defined in claim 33 wherein the processor is configured to command the system to supply the second pressure selected to make airflow during the current respiratory cycle as between the first naris and second naris substantially equal.

35. The system as defined in claim 33 wherein the processor is configured to command the system to supply the second pressure selected based on the attribute of the previous respiratory cycle.

36. The system as defined in claim 33 wherein the processor is configured to command the system to supply the first pressure during an inhalation of the current respiratory cycle, and wherein the processor is configured to command the system to supply the second pressure during the inhalation.

37. The system as defined in claim 36 further comprising wherein the processor is configured to command the system to supply a third pressure to the first naris during an exhalation of the current respiratory cycle, and wherein the processor is configured to command the system to supply a fourth pressure to the second naris during the exhalation, the fourth pressure selected to make more equal airflow carried as between the first naris and the second naris than if the third and fourth motor speeds were the same.

38. The system as defined in claim 33 wherein the processor is configured to command the system to supply the first pressure during an exhalation of the current respiratory cycle, and wherein the processor is configured to command the system to supply the second pressure during the exhalation.

39. The system as defined in claim 33 further comprising:
a first motor mechanically coupled to the first blower and electrically coupled to the processor, and wherein the processor commands the system to supply the first pressure by control of the first motor speed; and
a second motor mechanically coupled to the second blower and electrically coupled to the processor, and wherein the processor commands the system to supply the second pressure by control of the second motor speed.

40. The system as defined in claim 33 further comprising:
a first valve actuator;
a first valve coupled to the first blower and coupled to the first valve actuator, and wherein the processor commands the system to supply the first pressure by control of the position of the first valve;
a second valve actuator;
a second valve coupled to the first blower and coupled to the second valve actuator, and wherein the processor commands the system to supply the second pressure by control of the position of the second valve.

41. A nasal mask comprising:
a nose portion configured to cover and pneumatically seal around a patient's nose, the nose portion defining an internal cavity;
a supply hose fluidly coupled to the internal cavity and configured to couple to a positive airway pressure device;
a first airflow attribute sensing device mechanically coupled to the nose portion configured to be within inhaled airflow of a first naris of the patient when the nose portion positioned proximate to the patient's nose; and
a second airflow attribute sensing device mechanically coupled to the nose portion configured to be within inhaled airflow of a second naris of the patient when the nose portion positioned proximate to the patient's nose

42. The nasal mask as defined in claim 41 wherein the first and second airflow attribute sensing device are selected from the group: a thermocouple; a resistive thermal device; a piezoelectric temperature sensing device; or a sensing tube having an open end within the internal cavity and coupled to a pressure sensor.
